SEQUENCE LISTING (5 sequences)

SEQ ID NO: 1 - DNA, 110 bp, synthetic construct
  source 1..110: mol_type = other DNA
tcgagaaggtatattgctgttgacagtgagcgacccagattgaattccgagatatagtgaagccacagatgtatatctcggaattcaatctgggctgcctactgcctcgg

SEQ ID NO: 2 - DNA, 110 bp, synthetic construct
  source 1..110: mol_type = other DNA
tcgagaaggtatattgctgttgacagtgagcgagtgaggctgaaccgctataagtagtgaagccacagatgtacttatagcggttcagcctcacctgcctactgcctcgg

SEQ ID NO: 3 - DNA, 106 bp, synthetic construct
  source 1..106: mol_type = other DNA
tcgagaaggtatattgctgttgacagtgagcgaggatctgaagacacttctatagtgaagccacagatgtatagaagtgtcttcagatccctgcctactgcctcgg

SEQ ID NO: 4 - DNA, 110 bp, synthetic construct
  source 1..110: mol_type = other DNA
tcgagaaggtatattgctgttgacagtgagcgaggaattccttcctgaggattttagtgaagccacagatgtaaaatcctcaggaaggaattccctgcctactgcctcgg

SEQ ID NO: 5 - DNA, 110 bp, synthetic construct
  source 1..110: mol_type = other DNA
tcgagaaggtatattgctgttgacagtgagcgagcatgcttggaaggacatttgtagtgaagccacagatgtacaaatgtccttccaagcatgcctgcctactgcctcgg

(19) United States
(12) Patent Application Publication
(10) Pub. No.: US 2024/0279662 A1
(43) Pub. Date: Aug. 22, 2024

(54) A shRNA, its recombinant expression vector, transformant, anti-heart failure drug, preparation method, and use as drug thereof

(71) Applicant: Tongil Hospital, Tongji Medical College, Huazhong University of Science and Technology, Wuhan City (CN)

(72) Inventors: Daowen WANG, Wuhan City (CN); Huaping LI, Wuhan City (CN); Chen CHEN, Wuhan City (CN); Rong XIE, Wuhan City (CN)

(21) Appl. No.: 18/426,681

(22) Filed: Jan. 30, 2024

(30) Foreign Application Priority Data
Feb. 17, 2023 (CN) 202310137487.7

(51) Int. Cl. C12N 15/113; C12N 15/86

(52) U.S. Cl. CPC C12N 15/1136; C12N 15/86; C12N 2310/531; C12N 2750/14143; C12N 2830/008

(57) ABSTRACT

The present invention relates to a shRNA, its recombinant expression vector, transformant, anti-heart failure drug, preparation method, and use as drug thereof, belonging to a field of biomedicine. The present invention provides a shRNA whose DNA sequence is selected from a group consisting of SEQ ID NO. 1, SEQ ID NO. 3, and SEQ ID NO. 5. The present invention also provides a recombinant expression vector, transformant, anti-heart failure drug and its use as drug based on the shRNA, and provides a method for preparing the anti-heart failure drug. It's confirmed by animal experiments that the anti-heart failure drug provided by the present invention can significantly improve heart function of heart failure animals and play an effective anti-heart failure role.

### CROSS-REFERENCE TO RELATED APPLICATION

This application claims priority to and the benefit of Chinese patent Application No. 202310137487.7 filed Feb. 17, 2023. The entire contents of Chinese patent Application No. 202310137487.7 and the English translation of Chinese patent Application No. 202310137487.7 are incorporated herein by reference.

### SEQUENCE LISTING

This application contains references to amino acid sequences and/or nucleic acid sequences which have been submitted concurrently herewith as the sequence listing .xml file entitled “000007us_SequenceListing.xml”, file size 6,147 bytes, created on Jan. 27, 2024. The aforementioned sequence listing is hereby incorporated by reference in its entirety pursuant to 37 C.F.R. § 1.52(e)(5).

### TECHNICAL FIELD

The present invention belongs to a field of biomedicine, specifically relates to shRNA, its recombinant expression vector, transformant, anti-heart failure drug, preparation method, and pharmaceutical use.

### BACKGROUND

Cardiovascular disease has become a leading cause of death among Chinese people, and heart failure is a clinical syndrome in which different cardiovascular diseases develop to final stage. Although some new drugs have been applied in clinical practice in recent years, about 50% of heart failure patients still die within 5 years of diagnosis. Heart failure has a poor prognosis and high mortality rate, which is one of the main causes threatening human health and increasing medical burden.

At present, adenovirus and lentivirus are commonly used as expression vectors for gene therapy, but lentivirus is mostly modified from leukemia virus or HIV, and adenovirus vector has a short expression time and immunogenicity to body, both of which are not suitable for future clinical applications. The recombinant adeno-associated virus vector (rAAV) overcomes shortcomings that other gene expression vectors cannot overcome, has no immunogenicity, and can drive a long-term expression of target genes in vivo, thus it becomes a most promising vector for gene therapy.

Small nucleic acid drug refers to drug that can use small nucleic acid molecules such as siRNA, miRNA, and antisense nucleic acid ASO to specifically silence expression of disease genes, in order to cure specific diseases. Among them, double stranded RNA or hairpin structure RNA (shRNA) can be cleaved by endonucleases to form siRNA. Under action of helicase, it generates sense and antisense chains, and forms RNA induced silencing complexes to cleave and degrade target gene, thereby inhibiting expression of target gene. At present, multiple new small RNA drugs have been launched, such as the siRNA drug Incisiran for treating hyperlipidemia, and ASO drug Casimersen for treating Duchenne muscular dystrophy, which have great clinical application prospects. However, small RNA drug for treating heart failure in clinical practice are still research gap.

In the present invention, applicant has designed and developed a small RNA drug for rAAV delivery of short hairpin RNA (shRNA), which can significantly improve heart function and has a potential to treat heart failure.

### SUMMARY

Based on above objective problems and shortcomings in this field, inventors developed a small RNA drug rAAV-shRNA-AHF (AHF, anti-heart-failure), which delivered short hairpin RNA via rAAV, and conducted animal experiments to verify its effectiveness. It was found that rAAV-shRNA-shRNA-AHF can significantly improve mouse cardiac function and treat heart failure.

An above technical objectives of the present invention are achieved through the following technical solutions:

A shRNA, characterized in that, its DNA sequence is selected from a group consisting of SEQ ID NO.1, SEQ ID NO.3, and SEQ ID NO.5.

All of above shRNA can play a negative role in regulating myocardial hypertrophy marker ANP, which has a prospect and value of preparing anti-heart failure drugs. Among them, the shRNA of SEQ ID NO.1 has been verified by animal experiments to have anti-heart failure effects. Said DNA sequence refers to a DNA sequence in shRNA where the U base corresponds to the T base and all other bases are consistent with shRNA.

A recombinant expression vector, characterized in that, is an expression vector connecting shRNA; DNA sequence of shRNA is selected from a group consisting of SEQ ID NO.1, SEQ ID NO.3, and SEQ ID NO.5.

promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+).

A transformant, characterized in that, is a host transformed with a recombinant expression vector; the recombinant expression vector is an expression vector connecting shRNA; DNA sequence of shRNA is selected from a group consisting of SEQ ID NO.1, SEQ ID NO.3, and SEQ ID NO.5.

The host is selected from: virus, and/or cell;

Preferably, the virus is selected from adeno-associated virus;

Preferably, the cell is selected from 293 cell;

Preferably, promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+).

An anti-heart failure drug, comprising an active pharmaceutical ingredient, characterized in that, the active pharmaceutical ingredient comprises shRNA with DNA sequences selected from SEQ ID NO.1, SEQ ID NO.3, and SEQ ID NO.5.

The active pharmaceutical ingredient is selected from a group consisting of shRNA, recombinant expression vector, and transformant;
the shRNA has a sequence shown as SEQ ID NO.1; the transformant is a host containing a recombinant expression vector; the recombinant expression vector is a expression vector connecting shRNA with sequence shown as SEQ ID NO.1;

Preferably, the anti-heart failure drug further comprises: medicinal excipient;

Preferably, promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+).

A method of preparing an anti-heart failure drug, characterized in that, comprising the following step: preparing shRNA; DNA sequence of shRNA is selected from a group consisting of SEQ ID NO.1, SEQ ID NO.3, and SEQ ID NO.5.
said preparing is selected from a group consisting of synthesizing, amplifying, expressing, cloning, secreting, enriching, and expanding;

Preferably, said synthesizing refers to whole gene synthesis technology;

Preferably, said cloning refers to connecting shRNA to an expression vector;

Preferably, promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+).

A use of shRNA inpreparing anti-heart failure drug, characterized in that, DNA sequence of shRNA is selected from a group consisting of SEQ ID NO.1, SEQ ID NO.3, and SEQ ID NO.5.

The present invention provides a use of shRNA in preparing drug for treating heart failure.

Preferably, the expression vector is an adeno-associated virus expression vector pAAV D (+);

Preferably, the expression vector promoter is selected from: cardiomyocyte specific promoter tnt, α-MHC, MLC-2v, Desmin, or non-specific promoters H1, U6, CMV;

Preferably, the expression vector connecting shRNA-AHF is selected from a recombinant adeno-associated virus expression vector pAAV-D(+)-shRNA-AHF connecting the shRNA AHF sequence, or the recombinant adeno-associated virus expression vector pAAV-D(+)-tnt-shRNA-AHF, which sequentially connects to the myocardial specific promoter tnt and shRNA-AHF sequences;

Preferably, the host cell of the transformant is selected from 293 cell;

Preferably, the transformant transformed with the shRNA-AHF expression vector is selected from: recombinant adeno-associated virus rAAV-shRNA-AHF transformed with the recombinant adeno-associated virus expression vector pAAV-D(+)-shRNA-AHF, or recombinant adeno-associated virus rAAV-tnt-shRNA-AHF transformed with the recombinant adeno-associated virus expression vector pAAV-D(+)-tnt-shRNA-AHF;

Preferably, the shRNA-AHF sequence is shown as SEQ ID NO.1;

A recombinant expression vector with therapeutic effect on heart failure, characterized in that, is an expression vector connecting sequence of a gene with therapeutic effect on heart failure.

The expression vector is selected from: adeno-associated virus expression vector pAAV-D (+);

Preferably, the recombinant expression vector with therapeutic effect on heart failure is selected from recombinant adeno-associated virus expression vector pAAV-D(+)-shRNA-AHF connecting shRNA-AHF gene sequence.

A transformant with therapeutic effect on heart failure, characterized in that, is a host cell transformed with a recombinant expression vector with therapeutic effect on heart failure.

The host cell is selected from 293 cell;

Preferably, the transformant with therapeutic effect on heart failure is selected from recombinant adeno-associated virus rAAV-shRNA-AHF transformed with a recombinant adeno-associated virus expression vector pAAV-D(+)-shRNA-AHF1, or, recombinant adeno-associated virus rAAV-tnt-shRNA-AHF transformed with a recombinant adeno-associated virus expression vector pAAV-D(+)-tnt-shRNA-AHF;

Furthermore, the drug further comprises pharmaceutically acceptable excipient and/or reagent for buffering, synthesizing, and/or purifying the sequence fragments. A person skilled in the art can add various pharmaceutically acceptable adjuvant/excipient to the anti-heart failure drug of the present invention according to objective needs, and make various dosage forms for easy sales or promotion.

In further embodiments, the preparation method comprises the following steps: inserting a shRNA-AHF sequence fragment into an expression vector to prepare a recombinant plasmid capable of stably expressing shRNA-AHF.

In specific embodiments, the expression vector containing shRNA-AHF sequence fragment is adeno-associated virus expression vector pAAV-D (+).

To achieve a goal of gene therapy for heart failure, the present invention recombines shRNA-AHF sequence fragment with recombinant adeno-associated virus vector and obtains high titer that meet treatment requirements. It's confirmed by animal experiments that it can effectively improve heart function of heart failure mice. Therefore, based on above findings and results, the present invention provides a small RNA drug represented by shRNA-AHF for treating clinical heart failure.

The present invention relates to a drug for treating heart failure. The anti-heart failure drug involves construction and preparation method of a recombinant adeno-associated virus recombinant (rAAV-shRNA-AHF) of shRNA-AHF, and exerts therapeutic effects on heart failure by overexpressing the rAAV-shRNA-AHF. The present invention utilizes a chemical synthesis method to construct a pAAV-D(+)-shRNA-AHF expression plasmid, and uses three plasmid calcium phosphate co transfection methods to package, prepare, and purify recombinant adeno-associated virus containing target fragment. It's proven by animal experiments that the anti-heart failure drug provided by the present invention can significantly improve cardiac function of heart failure animals and play an effective anti-heart failure role.

The present invention designs and synthesizes sequence of shRNA-AHF, and successfully inserts it into eukaryotic expression vector pAAV-D (+) to form a recombinant plasmid pAAV-D(+)-shRNA-AHF. Afterwards, the following three plasmids: 1) pXX9 plasmid, 2) phelper plasmid, and 3) pAAV-D(+)-shRNA-AHF plasmid were transfected into 293 cell by using calcium phosphate co transfection method to prepare recombinant adeno-associated virus (rAAV9) that can express shRNA-AHF. After purification, titer was measured by using real-time PCR. The next step is to inject the packaged recombinant adeno-associated virus (rAAV-shRNA-AHF) of same serotype through the tail vein into a heart failure model mouse induced by Transverse Aortic Constriction surgery (TAC). It's shown by Ultrasound and catheter results that recombinant adeno-associated virus mediated shRNA-AHF can significantly improve cardiac function of TAC mice.

### DESCRIPTION OF DRAWINGS

In order to more clearly explaining the technical solution in embodiments of the present invention, a brief introduction will be made to drawings required in description of the embodiments. It is evident that the drawings in the following description are only some embodiments of the present invention. For those skilled in the art, other drawings can be obtained based on these drawings without creative work.

FIG. 1 shows a regulatory effect of the screened shRNA sequence on myocardial hypertrophy marker ANP in experiment example 1 of the present invention.

FIG. 2 is a schematic diagram of plasmid structure of pAAV-D(+)-shRNA-AHF prepared in experiment example 1 of the present invention; among them, tnt promoter represents cardiomyocyte specific promoter.

FIG. 3 shows an effect of rAAV-shRNA-AHF treatment on cardiac function of TAC mice monitored by Cardiac ultrasound and catheter in experiment example 2 of the present invention, wherein A is a bar graph of ejection fraction, B is a bar graph of shortened fraction, C is a bar graph of maximum rate of rise of left ventricular pressure during contraction, and D is a bar graph of the minimum rate of rise of left ventricular pressure during relaxation; it's shown by results that rAAV-shRNA-AHF significantly improved the cardiac systolic and diastolic function of TAC mice.

Meanings of each marker in drawings are listed as follows: rAAV-shNC represents TAC mice injected with a recombinant adeno-associated virus vector expressing control shRNA that specifically targets cardiomyocytes, rAAV-shRNA-AHF represents TAC mice injected with a recombinant adeno-associated virus vector of shRNA-AHF that specifically targets cardiomyocytes, Sham represents sham operated control mice, TAC represents TAC induced heart failure model mice.

### EMBODIMENTS

The technical solution of the present invention will be described clearly and completely in the following content by combining with specific examples and experimental examples. Obviously, the described experimental examples and examples are only a part of the present invention's experimental examples and examples, not all of them. Based on the experimental examples and examples of the present invention, all other examples obtained by a person skilled in the art without creative work fall within protection scope of the present invention.

Sources of instruments, equipment, reagents, consumables, and biomaterials involved in the following embodiments or experimental examples are as follows:

### (1) Instrument and Equipment

Nanodrop® ND-1000 nucleic acid analyzer, ABI 9700 PCR instrument, ABI 7900HT fluorescence real-time quantitative PCR instrument, Beckman X-15R low-temperature high-speed centrifuge;

### (2) Reagents and Consumables

RNasey Mini Kit (purchased from Qiagen), TRIZOL (purchased from Invitrogen), TaKaRa Agarose Gel DNA Purification Kit Ver. 2.0 (agarose gel DNA recovery kit, purchased from TaKaRa), E Z. N.A® Endo Free Plasmid Maxi Kit (Plasmid Extraction Kit, purchased from OMEGA company) and EasyPure Plasmid MiniPrep Kit (Plasmid Extraction Kit, purchased from Beijing TransGen Biotech company); an eukaryotic expression vector pAAV-D (+) was constructed and gifted by collaborating Professor Xiao Xiao. Core sequences of shRNA-1, shRNA-2, shRNA-3, shRNA-4, and shRNA-5 were synthesized by Sangon Biotech (Shanghai) Co., Ltd. The pAAV-shRNA-AHF sequence fragment was synthesized by Wuhan Keruist Biotechnology Co., Ltd.

### Sources of Biomaterials

C57 mice were purchased from Beijing Huafukang Company;

293T cells were purchased from ATCC Corporation in United States.

### Examples Group 1. shRNA of the Present Invention

This group of examples provides a shRNA. All examples in this group possess the following common features: DNA sequence of shRNA is selected from a group consisting of SEQ ID NO.1, SEQ ID NO.3, and SEQ ID NO.5.

In some embodiments, the shRNA is shRNA-AHF, and its DNA sequence is SEQ ID NO.1.

In some embodiments, the shRNA is shRNA-3, and its DNA sequence is SEQ ID NO.3.

In other embodiments, the shRNA is shRNA-5, and its DNA sequence is SEQ ID NO.5.

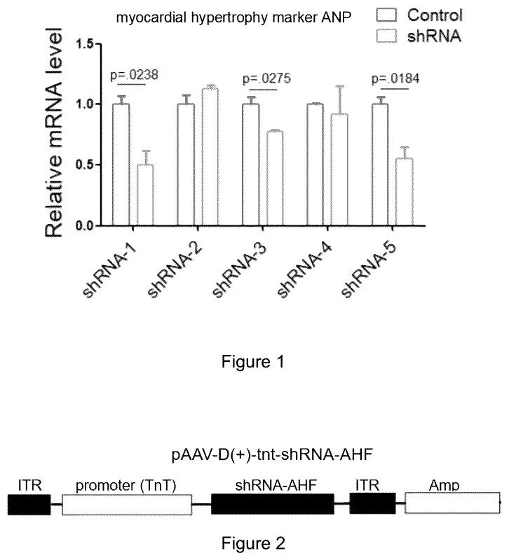

Above shRNA-AHF (shRNA-1), shRNA-3, and shRNA-5 can reduce expression of a marker of myocardial hypertrophy ANP (shRNA-1 has the most significant effect). Wherein, ShRNA-3 and shRNA-5 are targeted human sequences and cannot be used for mouse experiments; Due to limited time, human clinical experiments cannot be conducted in short term, so only results of cardiomyocyte experiments (as shown in FIG. 1) can be provided to demonstrate their protective function against cardiomyocyte.

All of above shRNA is designed and synthesized for the first time according to the present invention, and any act of amplifying, synthesizing, producing, manufacturing, selling, promising for sale, using, importing, exporting, secreting, expanding, enriching, connecting, transforming, cloning, expressing any of above shRNA, and/or any act of using any of above shRNA for pharmaceuticals or as pharmaceutical ingredients, and/or, any use of any of above shRNA for treatment falls within protection scope of the present invention.

### Examples Group 2. Recombinant Expression Vector of the Present Invention

This group of examples provide a recombinant expression vector. All examples of this group possess the following common features: the recombinant expression vector is an expression vector connecting shRNA; DNA sequence of shRNA is selected from a group consisting of SEQ ID NO.1, SEQ ID NO.3, and SEQ ID NO.5.

In specific embodiments, promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+).

All of above recombinant expression vectors are prepared for the first time according to the present invention, and any act of amplifying, synthesizing, producing, manufacturing, selling, promising for sale, using, importing, exporting, secreting, expanding, enriching, connecting, transforming, cloning, expressing above recombinant expression vectors, and/or any act of using above recombinant expression vectors for pharmaceuticals or as pharmaceutical ingredients, and/or, any use of above recombinant expression vector for treatment falls within protection scope of the present invention.

### Examples Group 3. Transformant of the Present Invention

This group of examples provides a transformant. All examples of this group possess the following common features: the transformant is a host transformed with a recombinant expression vector; the recombinant expression vector is an expression vector connecting shRNA; DNA sequence of shRNA is selected from a group consisting of SEQ ID NO.1, SEQ ID NO.3, and SEQ ID NO.5.

In some embodiments, the host is selected from: virus, and/or cell;

Preferably, the virus is selected from adeno-associated virus;

Preferably, the cell is selected from 293 cell;

Preferably, promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+).

All of above transformants are prepared for the first time according to the present invention. Any act of amplifying, synthesizing, producing, manufacturing, selling, promising for sale, using, importing, exporting, secreting, expanding, enriching, connecting, transforming, cloning, expressing above transformants, and/or any use of above transformants for pharmaceuticals or as pharmaceutical ingredients, and/or any use of above transformants for treatment, falls within protection scope of the present invention.

### Examples Group 4. Anti-Heart Failure Drug of the Present Invention

This group of examples provides an anti-heart failure drug. All examples of this group possess the following common features: the anti-heart failure drug comprises a active pharmaceutical ingredient, said active pharmaceutical ingredient comprises shRNA with DNA sequence selected from of SEQ ID NO.1, SEQ ID NO.3, and SEQ ID NO.5.

In some embodiments, the active pharmaceutical ingredient is selected from a group consisting of shRNA, recombinant expression vector, and transformant;

The shRNA has a sequence shown as SEQ ID NO.1; transformant is a host containing a recombinant expression vector; recombinant expression vector is an expression vector connecting shRNA of sequence shown as SEQ ID NO.1;

Preferably, the anti-heart failure drug further comprises: medicinal excipient; and/or, reagent for amplifying, synthesizing, producing, manufacturing, selling, promising for sale, using, importing, exporting, secreting, expanding, enriching, connecting, transforming, cloning, expressing shRNA as described in any one of the examples group 1, and/or, recombinant expression vectors as described in any one of the examples group 2, and/or, transformant as described in any one of the examples group 3;

A person skilled in the art, based on teachings of the present invention, add various pharmaceutically acceptable adjuvant/excipient to the anti-heart failure drug of the present invention according to production needs and practical requirements, and make various dosage forms for easy sales or promotion.

In specific embodiments, the medicinal excipient is selected from: solvents, projectiles, solubilizers, cosolvents, emulsifiers, colorants, adhesives, disintegrants, fillers, lubricants, wetting agents, osmotic pressure regulators, stabilizers, flow aids, flavor correction agents, preservatives, suspension aids, coating materials, aromatics, anti-adhesives, integrators, penetration enhancers, pH regulators, buffering agents, plasticizers, surfactants Foaming agents, defoamers, thickeners, packaging agents, moisturizers, absorbers, diluents, flocculants, anti-flocculants, filter aids, release blockers.

Preferably, promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+).

### Examples Group 5. Method of Preparing Anti-Heart Failure Drug of the Present Invention

This group of examples provides a method for preparing an anti-heart failure drug. All examples in this group possess the following common features: the method comprises the following steps: preparing shRNA; DNA sequence of shRNA is selected from a group consisting of SEQ ID NO.1, SEQ ID NO.3, and SEQ ID NO.5.

In specific embodiments, said preparing is selected from a group composed of synthesizing, amplifying, expressing, cloning, secreting, enriching, and expanding;

Preferably, the synthesis refers to whole gene synthesis technology;

Preferably, said cloning refers to connecting shRNA to an expression vector;

Preferably, promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+).

In further embodiments, the preparation method further comprises: combining or mixing active pharmaceutical ingredient with medicinal excipient.

A person skilled in the art, based on the teachings of the present invention, add various pharmaceutically acceptable adjuvant/excipient to the anti-heart failure drug of the present invention according to production needs and practical requirements, and make various dosage forms for easy sales or promotion.

In specific embodiments, the medicinal excipient is selected from: solvents, projectiles, solubilizers, cosolvents, emulsifiers, colorants, adhesives, disintegrants, fillers, lubricants, wetting agents, osmotic pressure regulators, stabilizers, flow aids, flavor correction agents, preservatives, suspension aids, coating materials, aromatics, anti-adhesives, integrators, penetration enhancers, pH regulators, buffering agents, plasticizers, surfactants Foaming agents, defoamers, thickeners, packaging agents, moisturizers, absorbers, diluents, flocculants, anti-flocculants, filter aids, release blockers.

### Examples Group 6. Use of shRNA as Drug of the Present Invention

This group of examples provides a use of shRNA in preparing anti-heart failure drugs. All examples of this group possess the following common features: DNA sequence of shRNA is selected from a group consisting of SEQ ID NO.1, SEQ ID NO.3, and SEQ ID NO.5.

In some embodiments, the anti-heart failure drug uses shRNA or recombinant expression vector or transformant as the active pharmaceutical ingredient;

In further embodiments, the anti-heart failure drug further comprises medicinal excipient;

In specific embodiments, the medicinal excipient is selected from: solvents, projectiles, solubilizers, cosolvents, emulsifiers, colorants, adhesives, disintegrants, fillers, lubricants, wetting agents, osmotic pressure regulators, stabilizers, flow aids, flavor correction agents, preservatives, suspension aids, coating materials, aromatics, anti-adhesives, integrators, penetration enhancers, pH regulators, buffering agents, plasticizers, surfactants Foaming agents, defoamers, thickeners, packaging agents, moisturizers, absorbers, diluents, flocculants, anti-flocculants, filter aids, release blockers.

In specific embodiments, the transformant is a host containing a recombinant expression vector; the recombinant expression vector is an expression vector connecting shRNA with sequence shown as SEQ ID NO.1, which can play a role in treating heart failure;

In the preferred embodiment, promoter of the expression vector is selected from: cardiomyocyte specific promoter tnt, α-MHC, MLC-2v, Desmin, or non-specific promoters H1, U6, CMV;

Preferably, the expression vector is selected from adeno-associated virus expression vector pAAV D (+);

Preferably, the host is selected from: virus, and/or cell;

Preferably, the virus is selected from adeno-associated virus;

Preferably, the cell is selected from 293 cell.

The following and experimental examples further describe the present invention:

### Experimental Example 1: Constructing Recombinant Adeno-Associated Virus

1. shRNA AHF Sequence Screening

During preliminary screening process, this invention designed several shRNA with potential anti-heart failure effects, as shown in SEQ ID NO.1 to SEQ ID NO.5, its core sequence was synthesized by Sangon Biotech (Shanghai) Co., Ltd. After cell experiment screening, it was found that shRNA-1 (i.e. shRNA-AHF) containing SEQ ID NO.1 sequence had the most significant effect in reducing a hypertrophy marker ANP (shRNA-3 and shRNA-5 were also able to reduce ANP) (FIG. 1). This invention further selected SEQ ID NO.1 for animal experiments as a representative of the experimental example. Construction described below is only some embodiments of the present invention. For those skilled in the art, similar embodiment results can be obtained based on these shRNA sequences without creative work.

2. Construction of pAAV-D (+) Vector

rAAV-shRNA-AHF double stranded nucleotides were synthesized using whole gene synthesis technology and cloned onto pAAV-D (+) vector (FIG. 2). The double stranded nucleotides were synthesized by Wuhan Keruist Biotechnology Co., Ltd. shRNA-AHF sequence is shown as SEQ ID NO.1.

### 3. Plasmid Transformation

Adding pAAV-D(+)-shRNA-AHF plasmid to 100 μl DH5a competent cell and placing it in ice for 30 minutes; after heating at 42° ° C. for 45 seconds, placing it in ice for 1 minute; adding 500 μL antibiotic free LB medium, oscillating at 37° C. and 100 rpm for 60 minutes; Cultivating on Amp+LB agar plate and selecting white monoclonal colonies for identification.

### 4. Plasmid Extraction at Small Scale

Selecting monoclonal colonies and adding them to 3 ml Amp+LB liquid culture medium. Incubating overnight at 37° C. and 280 rpm. Using EasyPure Plasmid MiniPrep Kit from Beijing TransGen Biotech Company to extract plasmids. The specific steps are as follows: 1. Taking 1.5 ml of overnight cultured bacteria and centrifuging at 10000 g for 1 minute, trying to suck up supernatant as much as possible; 2. adding 250 μL colorless solution RB (containing RNase A), shaking suspension bacterial precipitation; 3. adding 250 μl blue solution LB and mixing by gently flipping it up and down 4-6 times, allowing bacterial cells to fully lyse and form a bright blue solution; 4. adding 350 μl yellow solution NB and mixing gently 5 to 6 times until a compact yellow agglutination is formed, and placing it at room temperature for 2 minutes; 5. centrifuging at 15000 g for 5 minutes, carefully absorbing supernatant and adding it to adsorption column; 6. centrifuging 15000 g for 1 minute and discarding effluent; 7. adding 650 μl of solution WB and centrifuging at 15000 g for 1 minute and discarding effluent; 8. centrifuging at 15000 g for 2 minutes to thoroughly remove residual WB; 9. placing adsorption column in a new Ep tube and adding 20 μl of preheat EB at 70° C. in center of the column and placing it at room temperature for 1 minute; 10. centrifuging at 10000 g for 1 minute, eluting DNA, and storing the eluted DNA at 20° C.

### 5. Plasmid Extraction at Large Scale

Preparing a 1 L sterile conical flask, adding 300 ml sterile LB medium, and adding ampicillin solution until final concentration is 100 μg/ml. Adding 50 μl of the required plasmids (pXX9, phelper, pAAV-D(+)-shNC, pAAV-D(+)-shRNA-AHF) respectively and culturing overnight at 280 rpm and 37° C. According to instructions of Endo-Free Plasmid Maxi Kit from OMEGA company, extracting plasmid. The specific steps are as follows: 1. centrifuging at 5000 g at room temperature for 10 minutes to collect bacteria; 2. discarding culture medium, adding 10 ml of Solution I/RNase A mixture, and resuspending completely with vortex shaking; 3. adding 10 ml of Solution II to the resuspended mixture, mixing by gently inverting 10 to 15 times, then placing at room temperature

6. rAAV Mediated Virus Packaging

293T cells grew to 90%, and 12 hours before calcium phosphate transfection, each culture dish was replaced with 12-15 ml of fresh culture medium (including serum). Firstly adding Calcium chloride (CaCl2) to a 50 ml centrifuge tube, then adding plasmid to form a Ca-DNA mixture, and mixing thoroughly. Dropping 2× HEBS BUFFER slowly to Ca-DNA mixture to form a Ca-DNA-P mixture. Adding 2× HEBS while shaking centrifuge tube to fully mix and form calcium phosphate particles. After 8-12 hours, replacing with 18-20 ml serum-free medium. After 72 hours, discarding medium and washing it 3 times with PBS. Adding 1 ml of Tris+NaCl (pH 8.5) to each culture dish, scraping cells with a scraper, collecting them in a clean centrifuge tube, and freezing at 80° C.

### 7. Virus Purification

Taking out cells that have been frozen at 80° C., thawing and dissolving them at 37° C., repeatedly freezing and thawing 4 times, centrifuging at 8000 g for 15 minutes, and transferring supernatant to a clean centrifuge tube, and discarding cell precipitate. Mixing anhydrous ethanol pre-cooled at 20° C. with rAAV in a 3:1 volume ratio, placing in a refrigerator at 20° C. for 2 hours, centrifuging at 4° C. and 13000 rpm for 15 minutes, and discarding supernatant; after evaporation of ethanol, adding the corresponding volume of Tris+NaCl (pH 8.5) to dissolve precipitate. Using a Millipore small filter (0.22 μm) for filtering.

### 8. Virus Titer Determination

Sample processing:
RAAV virus solution 40 μL
Protease K (20 mg/ml) 5 μL
55° C., reaction for 1 hour;
Phenol: chloroform: isoamyl alcohol 45 μL
4° C., centrifuge at 12000 g for 5 minutes to recover a aqueous phase;
Chloroform 45 μL
4° C., centrifuge 12000 g for 5 minutes to recover a aqueous phase.
Real-time PCR:
Primer 1(10 μm) 0.4 μl
Primer 2(10 μm) 0.4 μl
SYBR Green I Mix 10 μl
ddH2O 8.2 μl
Template 1 μl
95° C. 30 sec (95° C. 5 sec, 60° C. 5 sec, 72° C. 20 sec)×40 cycles—Melting Curve

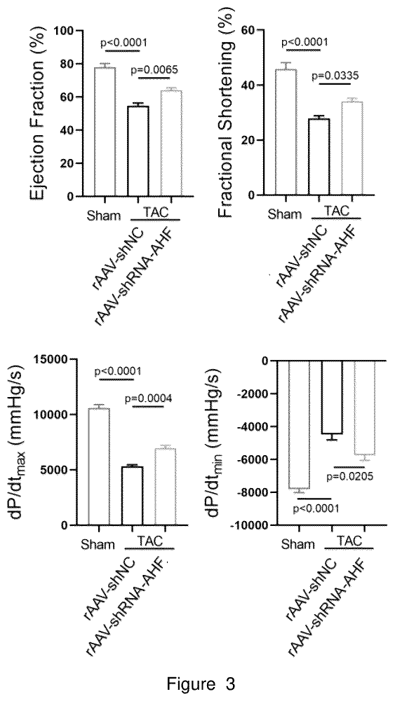

Experimental example 2. taking recombinant adeno-associated virus of rAAV9 type expressing shRNA AHF as an example to test its therapeutic effect on heart failureUsing 8-week-old C57 mice, injecting rAAV-tnt-shRNA-AHF and control rAAV-tnt-shNC virus into tail vein, with a viral titer of 1×1011PFU per mouse, performing TAC surgery 2 weeks later: Transverse Aortic Constriction (TAC) surgery. At the end of the experiment (4 weeks later), measuring mouse cardiac function with the following method: using an ultrasound instrument equipped with a 30 MHz high-frequency probe. After anesthetizing mice with isoflurane, placing the mice supinely on a testing platform, and collecting two-dimensional images of left ventricle along the horizontal short and long axis sections of left ventricular papillary muscle near sternum of the mice. At the same time, obtaining M-mode ultrasound images of more than 5 consecutive cardiac cycles under guidance of two-dimensional images. Based on the collected images, software analysis was used to obtain cardiac hemodynamic indicators for echocardiography, After analysis by relevant software, the following indicators were calculated: heart rate (HR), left ventricular diastolic diameter (LVIDd), left ventricular systolic diameter (LVIDs), left ventricular posterior wall diastolic thickness (LVPWd), left ventricular posterior wall systolic thickness (LVPWs) Interventricular septal thickness (IVSd), interventricular septal thickness (systole, IVSs), ejection fraction (EF), and fractional shortening (FS). It's shown by results that rAAV-shRNA-AHF treatment significantly improved cardiac function of TAC mice (FIG. 3).

What is claimed is:

1. A shRNA, characterized in that, its DNA sequence is selected from a group consisting of SEQ ID NO.1, SEQ ID NO.3, and SEQ ID NO.5.

2. An anti-heart failure drug, comprising an active pharmaceutical ingredient, characterized in that, the active pharmaceutical ingredient comprises shRNA with DNA sequences selected from SEQ ID NO.1, SEQ ID NO.3, and SEQ ID NO.5.

3. The anti-heart failure drug according to claim 2, characterized in that, the active pharmaceutical ingredient is selected from a group consisting of shRNA, a recombinant expression vector, and a transformant;
the shRNA has a sequence shown as SEQ ID NO.1.

4. The anti-heart failure drug according to claim 3, characterized in that, the transformant is a host containing a recombinant expression vector; the recombinant expression vector is an expression vector connecting shRNA with sequence shown as SEQ ID NO.1.

5. The anti-heart failure drug according to claim 3, characterized in that, said an anti-heart failure drug further comprising: medicinal excipient.

6. The anti-heart failure drug according to claim 4, characterized in that, promoter of the expression vector is selected from: cardiomyocyte specific promoter tnt, α-MHC, MLC-2v, Desmin, or non-specific promoters H1, U6, CMV;
and/or, the expression vector is selected from adeno-associated virus expression vector pAAV D (+).

7. The anti-heart failure drug according to claim 4, characterized in that, the host is selected from: virus and/or cell;
and/or, the virus is selected from adeno-associated virus;
and/or, the cell is selected from 293 cell.

8. A method of preparing an anti-heart failure drug, characterized in that, comprising the following step: preparing shRNA; DNA sequence of shRNA is selected from a group consisting of SEQ ID NO.1, SEQ ID NO.3, and SEQ ID NO.5.

9. The method of preparing an anti-heart failure drug according to claim 8, characterized in that, also comprising the following steps: said preparing is selected from a group consisting of synthesizing, amplifying, expressing, cloning, secreting, enriching, and expanding.

10. The method of preparing an anti-heart failure drug according to claim 8, characterized in that, said synthesizing refers to whole gene synthesis technology;
and/or, said cloning refers to connecting shRNA to an expression vector;
and/or, promoter of the expression vector is selected from: cardiomyocyte specific promoter tnt, α-MHC, MLC-2v, Desmin, or non-specific promoters H1, U6, CMV;
and/or, the expression vector is selected from adeno-associated virus expression vector PAAV D (+).
